(19) United States
(12) Patent Application Publication
(10) Pub. No.: US 2017/0368235 A1
(43) Pub. Date: Dec. 28, 2017

(54) PHARMACEUTICAL COMPOSITIONS AND DEVICE METHODS FOR TREATMENT OF PROLIFERATIVE DISEASES

(71) Applicant: Thomas Q. Dinh, Minnetonka, MN (US)

(72) Inventor: Thomas Q. Dinh, Minnetonka, MN (US)

(21) Appl. No.: 15/694,921

(22) Filed: Sep. 4, 2017

Related U.S. Application Data
(62) Division of application No. 14/797,068, filed on Jul. 10, 2015, now abandoned.
(60) Provisional application No. 62/023,872, filed on Jul. 12, 2014.

(51) Int. Cl. A61L 29/16; A61L 29/08; A61M 25/10

(52) U.S. Cl. CPC A61L 29/16; A61L 29/085; A61L 2420/06; A61L 2300/802; A61L 2300/45; A61L 2300/436; A61L 2300/416; A61M 2025/105; A61L 2300/606

(57) ABSTRACT

A method for treating proliferative diseases by delivering a combination of at least two pharmaceutically active agents to a diseased area or tissue comprising a coating layer of two hydrophobic drugs applied to an exterior surface of a device or a substrate wherein the first pharmaceutically active agent is selected from a group consisting of mTor inhibitors and the second pharmaceutically active agent is selected from a group of consisting of NF-kβ inhibitors. Further a method for treating proliferative diseases by delivering a combination of at least two pharmaceutically active agents to a diseased area or tissue comprising:
a coating layer of two hydrophobic drugs applied to an exterior surface of a medical device or substrate and a polymer blend carrier for the pharmaceutically active agents.

### CROSS-REFERENCE TO PENDING APPLICATIONS

This application is a divisional of, and therefore claims priority to, U.S. Utility application Ser. No. 14/797,068 filed Jul. 10, 2015. The contents of file Ser. No. 14/797,068 are hereby incorporated in their entirety. application Ser. No. 14/797,068 claims the benefit of U.S. Provisional Application No. 62/023,872, filed Jul. 12, 2014.

### FIELD OF THE INVENTION

The present invention discloses various embodiments related to drug coated devices, especially balloon catheters, and their use for delivering at least two or more therapeutic agents to a diseased tissue or an obstructive conduit inside the body.

### BACKGROUND OF THE INVENTION

Proliferative diseases such as peripheral vascular disease (PVD) is a nearly pandemic condition that has the potential to cause the loss of a limb or even the loss of life. Peripheral vascular disease manifests as insufficient tissue perfusion caused by existing atherosclerosis that may be acutely compounded by either emboli or thrombi. Many people live daily with peripheral vascular disease; however, in settings such as acute limb ischemia, this pandemic disease can be life threatening and can require emergency intervention to minimize morbidity and mortality.

PVD afflicts an estimated 20 million people in the US and Europe and the lack of effective current solutions results in over 250,000 amputations per year. Balloon angioplasty is often used to treat restricted vessels due to PVD but suffers from a greater than 40% restenosis rate because of smooth muscle cell proliferation caused by the procedure and the healing response of the injured site. Stents are largely ineffective in the periphery because of the mechanical challenges associated with normal flexing of the leg and other pressures as a result of daily activities.

Over the last decade, many local drug delivery systems have been developed for the treatment and/or the prevention of restenosis after balloon angioplasty or stenting. One example of a local delivery system is a drug eluting stent (DES). The stent is coated with a polymer into which a drug is impregnated. When the stent is inserted into a blood vessel, the drug is slowly released from the polymer or drug carrier. The slow release of the drug, which takes place over a period of a few weeks, has been reported as one of the main advantages of using DES. The current generation drug eluting stents usually contain the drugs Paclitaxel or rapamycin. While drug-eluting stents were initially shown to be an effective technique for reducing and preventing restenosis, recently their efficacy and safety have been questioned due to late thrombosis and a lack of tissue healing. This late thrombosis is a major complication and life-threatening side effect of DES devices.

The paclitaxel drug-coated balloon (DCB) is an emerging device in percutaneous coronary intervention (PCI) developed to circumvent some of the limitations faced by drug-eluting stents (DES) mentioned above. DCB are semi-compliant angioplasty balloons covered with an anti-restenotic drug that is rapidly released locally into the vessel wall during balloon angioplasty.

Various companies manufacture paclitaxel coated DCB because of their lipophilicity and tissue retention characteristics. These paclitaxel DCB differ in drug-delivery technology and excipients used, thereby resulting in differences in specific elution kinetics and tissue retention. These mechanistic differences are not well understood, however, and their clinical significance is even less clear. Angioplasty balloons that are coated with the drug Taxol usually contain about 2-3 micrograms per square millimeter of balloon surface. For a typical drug coated balloon for treatment of PVD, this would amount to a few milligrams of drug. With more than 90 percent of drug washed out during the procedure, this would present an unsafe and lethal dose of Taxol downstream of the affected diseased vessel. Taxol is a cytotoxic drug and has been implicated in late stent thrombosis because high concentration of Taxol residues in the artery wall can prevent proper healing by inhibiting the endothelization.

Thus, there is a need to identify a drug formulation that can provide better healing to the treated diseased vessel and internal tissues. Since restenosis and healing processes are affected by multiple factors, it is desirable to identify a combination of at least two pharmaceutical agents that can act synergistically to combat different molecular pathways in the process of restenosis and wound healing. Pharmaceutical compounds that can act synergistically would allow one to treat the disease at a much lower dose as compared to one drug alone. In addition, the combination can lower the total dose on a medical device significantly, thus lowering the risk of drug wash out and improving the safety of the treatment and device for the patient.

### SUMMARY OF THE INVENTION

The present invention discloses a coating formulation of a combination of two or more pharmaceutical agents on the exterior surface of a medical device, particularly of a balloon catheter or a stent or any conduit that can be placed within the body lumen. The formulation of the coating consists of at least two pharmaceutical agents that act synergistically in inhibiting smooth muscle cell proliferation while simultaneously providing a pro-healing outcome. It is also the subject of this invention to disclose pharmaceutical agents that can act as permeation enhancers that allow other drugs to penetrate tissues more effectively. In the present invention, at least one pharmaceutical in the formulation can act as a primer for a uniform coating of the drug mixture onto the surface of the device, particularly drug coated balloons. Furthermore, the coatings according to embodiments of the present invention facilitate rapid drug elution off of the surface of the device and superior permeation of drug into tissues at a disease site to treat disease. This is accomplished by using a pharmaceutical agent that has a strong affinity for tissue staining, also stated has having tissue-staining properties. Thus, coatings according to embodiments of the present invention provide an enhanced rate of absorption of the lipophilic therapeutic agents in diseased tissues of the vasculature, other body lumen, or organ tissues.

In embodiments of the present invention, the coated device reduces cell proliferation and enhances the healing process of a body lumen. As a result, better clinical outcomes can be achieved in the long term.

In embodiments of the present invention, at least one active agent is an mTor inhibitor and the other pharmaceutical agent is an NF-kβ inhibitor, whereas the NF-kβ inhibitor also acts as a primer for the coating.

In embodiments of the present invention, the mTor inhibitor is rapamycin or rapamycin analogs and the other NF-kβ inhibitor is curcumin or curcumin analogs, whereas curcumin or its analogs also act as a primer for the coating and a coloring agent.

In some embodiments of the invention, the mTor inhibitor is administered in combination with an NF-kβ inhibitor effective for treatment of cell proliferation or restenosis in the region of the lumen where administered. The NF-kβ inhibitor can include curcumin, sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, and cycloepoxydon tepoxalin and mixtures thereof in various embodiments.

In some embodiment formulations the mTor inhibitor is pegylated. The pegylated mTor inhibitor includes pegylated Interferon-α and Interferon-β in some embodiments.

In embodiments of the present invention, the weight ratio of the mTor inhibitor to the NF-kβ inhibitor is from 1:1 to 100:1.

In embodiments of the present invention, the weight ratio of rapamycin or rapamycin analogs to curcumin or curcumin analogs is from 1:1 to 100:1.

In embodiments of the present invention, at least one active agent is an mTor inhibitor and the other pharmaceutical agent is an NF-kβ inhibitor, wherein the combined drug loading of both pharmaceutical agents is from 0.1 micrograms per square millimeter to 10 micrograms per square millimeter.

In embodiments of the present invention, at least one active agent is rapamycin or rapamycin analogs and the other pharmaceutical agent is curcumin or curcumin analogs, wherein the combined drug loading of both pharmaceutical agents is from 0.1 micrograms per square millimeter to 10 micrograms per square millimeter.

In embodiments of the present invention, the NF-kβ inhibitor has dual functionality as a tissue-staining agent, also stated as having tissue-staining properties, and a tissue permeation enhancer.

In embodiments of the present invention, curcumin or curcumin analogs have dual functions as a tissue staining agent as well as a tissue permeation enhancer.

In embodiments of the present invention, at least one active agent is an mTor inhibitor and the other pharmaceutical agent is an NF-kβ inhibitor, whereas the NF-kβ inhibitor has an absorption wavelength in the Visible region of the UV-Vis spectrum.

In embodiments of the present invention, mTor inhibitor is rapamycin or rapamycin analogs and the NF-kβ inhibitor is curcumin or curcumin analogs, whereas curcumin or curcumin analogs have an absorption wavelength in the Visible region of the UV-Vis spectrum.

In embodiments of the present invention, at least one active agent is an mTor inhibitor and the other pharmaceutical agent is an NF-kβ inhibitor and an additional permeation enhancer, whereas the permeation enhancer is selected from the group consisting of dodecyl methyl sulfoxide (DMSO), citric acid, and combinations thereafter.

In embodiments of the present invention, at least one active agent is rapamycin or rapamycin analogs and the other pharmaceutical agent is curcumin or curcumin analogs and an additional permeation enhancer, whereas the permeation enhancer is selected from the group consisting of dodecyl methyl sulfoxide (DMSO), citric acid, and combinations thereof.

In embodiments of the present invention, at least one active agent is an mTor inhibitor and the other pharmaceutical agent is an NF-kβ inhibitor and an additional permeation enhancer, whereas the permeation enhancer is selected from the group of Nitric Oxide donors (NO), whereas the NO donor is selected from a group of S-nitrosothiols consisting of S-nitroso-N-acetylamine (SNAP), S-nitrosoglutathione (SNOGLU) and S-nitroso-N-valerylpenicillamine (SNVP) and a group of Diazeniumdiolates (NONOates) consisting of Diethyamino NONOate (DEA-NO), PROLI/NO, SPER/NO and V-PYRRO/NO.

In embodiments of the present invention, at least one active agent is rapamycin or rapamycin analogs and the other pharmaceutical agent is curcumin or curcumin analogs and an additional permeation enhancer, whereas the permeation enhancer is selected from the group of Nitric Oxide (NO) donors, whereas the NO donor is selected from a group of S-nitrosothiols consisting of S-nitroso-N-acetylamine (SNAP), S-nitrosoglutathione (SNOGLU) and S-nitroso-N-valerylpenicillamine (SNVP) and a group of Diazeniumdiolates (NONOates) consisting of Diethyamino NONOate (DEA-NO), PROLI/NO, SPER/NO and V-PYRRO/NO.

In embodiments of the present invention, at least one active agent is an mTor inhibitor and the other pharmaceutical agent is an NF-kβ inhibitor and an additional permeation enhancer, whereas the permeation enhancer is sodium nitroprusside (SNP).

In embodiments of the present invention, at least one active agent is rapamycin or rapamycin analogs and the other pharmaceutical agent is curcumin or curcumin analogs and an additional permeation enhancer, whereas the permeation enhancer is sodium nitroprusside (SNP).

In embodiments of the present invention, at least one active agent is an mTor inhibitor and the other pharmaceutical agent is an NF-kβ inhibitor and an additional polymer blend, whereas the polymer blend is not miscible.

In embodiments of the present invention, at least one active agent is an mTor inhibitor and the other pharmaceutical agent is an NF-kβ inhibitor and an additional polymer blend, whereas the polymer blend is a mixture of hydrophilic polyurethane and polyacrylic acid, wherein the polymer blend has a ratio by weight of the polyurethane to the polyacrylic from 1:1 to 10:1.

In embodiments of the present invention, at least one active agent is rapamycin or rapamycin analogs and the other pharmaceutical agent is curcumin or curcumin analogs and an additional polymer blend, whereas the polymer blend is not miscible.

In embodiments of the present invention, at least one active agent is rapamycin or rapamycin analogs and the other pharmaceutical agent is curcumin or curcumin analogs and an additional polymer blend, whereas the polymer blend is a mixture of hydrophilic polyurethane and polyacrylic acid; the polymer blend has a ratio by weight of the polyurethane polymer to the polyacrylic polymer from 1:1 to 10:1

Some embodiments of the present invention provide methods for treating a diseased tissue to prevent cell proliferation and tissue inflammation. The methods can include delivering a therapeutically effective amount of an anti-inflammatory agent (AA) and a therapeutically effective amount of an anti-proliferative inhibitor (AI) into the diseased tissue. In some methods, the AA and the AI are delivered from a controlled release carrier, which can include the AA and the AI being controllably released from immiscible polymer blends, in various embodiments. The AA and the AI can be controllably released through a diffusion mechanism in some methods.

In some embodiments, the AI is rapamycin and the AA is curcumin or one of the group of anti-inflammatory drugs including sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, and cycloepoxydon tepoxalin and mixtures thereof in various embodiments.

In one embodiment, the present invention relates to a medical device for delivering a combination of at least two therapeutic agents to a tissue for reducing cell proliferation and inflammation, the device comprising a coating of the drug mixture on an exterior surface of the medical device. The device includes one of a balloon catheter, a stent, and a stent graft, prosthesis such as a heart valve, a suture, a mesh, and a patch. In addition, the proliferative tissue mentioned in this invention includes vascular tissues of both coronary and peripheral vasculatures and conduits in the body such as the urinary tract, prostate, ovaries, fistula tracts, and the uterus.

In some embodiment of this invention, the coating solution of drug mixture is designed to adhere to polymeric materials such as polyethylene, polypropylene, nylon, PET (polyethylene terephthalates), Dacron, PLGA, PLLA, polycaprolactone and other metals such as stainless steel, cobalt, tantalum, nitinol, MP-35 alloys, and platinum.

In one embodiment, the drug layer of the medical device consists essentially of therapeutic agents in various ratios; with the anti-inflammatory agent having a lower weight ratio than the anti-proliferative agent.

In one embodiment, the present invention relates to a medical device for delivering a combination of at least two therapeutic agents to the pudendal artery to treat erectile dysfunction, the device comprising a coating of the drug mixture on an exterior surface of the medical device. The device includes one of a balloon catheter, a stent, and a stent graft. The combination of pharmaceutical agents includes the mTor inhibitor that is rapamycin or rapamycin analogs and the other NF-kβ inhibitor that is curcumin or curcumin analogs, whereas curcumin or its analogs also act as a primer for the coating and a coloring agent.

In one embodiment, the permeation enhancer enhances penetration and absorption of the therapeutic agents in tissue. In another embodiment, the permeation enhancer enhances release of the combined therapeutic agents from the surface of the medical device. In another embodiment, the permeation enhancer is soluble in the same solvent as the therapeutics agents, preferably tetrahydrofuran or methylene chloride.

In one embodiment of the medical device, the device is capable of delivering the combination of the therapeutic agents to the tissue in about 0.1 to 10 minutes, or preferably from about 0.1 to 2 minutes.

In one embodiment, the present invention relates to a balloon catheter for delivering a combination of at least two therapeutic agents to a blood vessel for reducing stenosis, the balloon catheter comprising a coating layer of the drug mixture on its exterior surface.

In one embodiment, the present invention relates to a balloon catheter for delivering a combination of an mTor inhibitor and an NF-kβ inhibitor to a blood vessel for reducing stenosis, the catheter comprising a coating layer of the drug mixture of these two inhibitors on the exterior surface of a balloon.

In one embodiment, the present invention relates to a balloon catheter for delivering a combination of an mTor inhibitor and an NF-kβ inhibitor to a blood vessel for reducing stenosis, wherein the mTor inhibitor is rapamycin or rapamycin analogs and the NF-kβ inhibitor is curcumin or curcumin analogs, the catheter comprising a coating layer of the drug mixture of these two inhibitors on the exterior surface of a balloon.

In another embodiment of the balloon catheter, the coating layer comprises a combination of at least two therapeutic agents and an additional permeation enhancer.

In one embodiment, the present invention relates to a balloon catheter for delivering a combination of an mTor inhibitor and an NF-kβ inhibitor to a blood vessel for reducing stenosis, wherein the mTor inhibitor is rapamycin or rapamycin analogs and the NF-kβ inhibitor is curcumin or curcumin analogs, and a permeation enhancer, the catheter comprising a coating layer of the drug mixture of these two inhibitors and the permeation enhancer on the exterior surface of a balloon.

In one embodiment, the present invention relates to a balloon catheter for delivering a combination of an mTor inhibitor and an NF-kβ inhibitor to a blood vessel for reducing stenosis, wherein the mTor inhibitor is rapamycin or rapamycin analogs and the NF-kβ inhibitor is curcumin or curcumin analogs, and a permeation enhancer, whereas the permeation enhancer is selected from a group consisting of dodecyl methyl sulfoxide (DMSO), citric acid, and combinations thereafter.

In one embodiment, the present invention relates to a balloon catheter for delivering a combination of an mTor inhibitor and an NF-kβ inhibitor to a blood vessel for reducing stenosis, wherein the mTor inhibitor is rapamycin or rapamycin analogs and the NF-kβ inhibitor is curcumin or curcumin analogs, and a permeation enhancer, whereas the permeation enhancer is selected from the group of Nitric Oxide donors (NO) and the NO donor is selected from a group of S-nitrosothiols consisting of S-nitroso-N-acetylamine (SNAP), S-nitrosoglutathione (SNOGLU) and S-nitroso-N-valerylpenicillamine (SNVP) and a group of Diazeniumdiolates (NONOates) consisting of Diethyamino NONOate (DEA-NO), PROLI/NO, SPER/NO and V-PYRRO/NO consisting of dodecyl methyl sulfoxide (DMSO), citric acid, and combination thereafter.

In one embodiment, the present invention relates to a balloon catheter for delivering a combination of an mTor inhibitor and an NF-kβ inhibitor to a blood vessel for reducing stenosis, wherein the mTor inhibitor is rapamycin or rapamycin analogs and the NF-kβ inhibitor is curcumin or curcumin analogs, and a permeation enhancer, whereas the permeation enhancer is sodium nitroprusside.

In one embodiment, the balloon catheter is capable of delivering the combination of the therapeutic agents to the blood vessel in about 0.1 to 2 minutes. In another embodiment, the balloon catheter is capable of delivering the combination of the therapeutic agents to the blood vessel in about 0.1 to 1 minute.

In one embodiment of the balloon catheter, the concentration of the combined therapeutic agents in the coating layer is from 0.3 to 20 micrograms per square millimeter. In another embodiment, the concentration of the combined therapeutic agents in the coating layer is from 0.5 to 10 micrograms per square millimeter.

In yet a further embodiment, the present invention relates to a method for treating a proliferative condition of a body lumen or part of an organ after a surgical or interventional procedure comprising delivering a formulation of the pharmaceutical composition disclosed in this invention to the surgical site by injection with a catheter, wherein the mTor inhibitor is rapamycin or rapamycin analogs and the other NF-kβ inhibitor is curcumin or curcumin analogs,

In yet a further embodiment, the present invention relates to a pharmaceutical composition for treating various types of cancers locally including cancers of the breast, lung, uterus, ovaries, pancreas, liver, prostate, bladder, brain, colon and skin cancer, wherein the composition comprises at least an mTor inhibitor and the other pharmaceutical agent is an NF-kβ inhibitor, whereas the NF-kβ inhibitor also act as permeation enhancers.

In yet a further embodiment, the present invention relates to a pharmaceutical composition for treating various types of cancers locally including cancers of the breast, lung, uterus, ovaries, pancreas, liver, prostate, bladder, brain, colon and skin cancer, wherein the mTor inhibitor is rapamycin or rapamycin analogs and the other pharmaceutical agent is curcumin or curcumin analogs, whereas curcumin or curcumin analogs also acts as a permeation enhancer.

In yet a further embodiment, the present invention relates to a pharmaceutical composition for treating various types of hyperplasia locally including benign prostate hyperplasia (BPH) and endometrial hyperplasia, wherein the composition comprises at least an mTor inhibitor and the other pharmaceutical agent is an NF-kβ inhibitor, whereas the NF-kβ inhibitor also acts as a permeation enhancer.

In yet a further embodiment, the present invention relates to a pharmaceutical composition for treating various types of hyperplasia locally including benign prostate hyperplasia (BPH) and endometrial hyperplasia, wherein the composition comprises rapamycin or rapamycin analogs and curcumin or curcumin analogs, whereas curcumin or curcumin analogs also acts as permeation enhancers.

In yet a further embodiment, the present invention relates to a pharmaceutical composition for preventing inflammation and angiogenesis of various diseased tissues including bone joints of the knee, foot, ankle, hand, finger, lumbar, cervical, thoracic, and the hip, wherein the composition comprises at least an mTor inhibitor and the other pharmaceutical agent is an NF-kβ inhibitor, whereas the NF-kβ inhibitor also acts as a permeation enhancer.

In yet a further embodiment, the present invention relates to a pharmaceutical composition for preventing inflammation and angiogenesis of various diseased tissues including bone joints of the knee, foot, ankle, hand, finger, lumbar, cervical, thoracic, and the hip, wherein the mTor inhibitor is rapamycin or rapamycin analogs and the NF-kβ inhibitor is curcumin or curcumin analogs, whereas curcumin or curcumin analogs also act as permeation enhancers.

In yet a further embodiment, the present invention relates to a pharmaceutical composition for preventing inflammation and infection of various diseased tissues of the ear, nose and throat (ENT), wherein the composition comprises at least an mTor inhibitor and the other pharmaceutical agent is an NF-kβ inhibitor, whereas the NF-kβ inhibitor also acts as a permeation enhancer.

In yet a further embodiment, the present invention relates to a pharmaceutical composition for preventing inflammation and infection of various diseased tissues of the ear, nose and throat (ENT), wherein the mTor inhibitor is rapamycin or rapamycin analogs and the NF-kβ inhibitor is curcumin or curcumin analogs, whereas curcumin or curcumin analogs also acts as a permeation enhancer.

In one embodiment, the present invention relates to a process of producing a drug coated balloon catheter. In one aspect of this embodiment, the process comprises preparing a solution comprising an organic solvent, an mTor inhibitor and an NF-kβ inhibitor, and then applying the solution to the balloon catheter, and evaporating the solvent.

Many embodiments of the present invention are particularly useful for treating vascular disease, proliferative disease, inflammatory disease, cancer, and for reducing stenosis and late luminal loss, or are useful in the manufacture of devices for that purpose.

It is understood that both the foregoing general description and the following detailed description are exemplary and explanatory only and are not restrictive of the present invention as claimed.

### BRIEF DESCRIPTION OF THE DRAWINGS

FIG. 1 shows an example of a drug-coated balloon of a balloon catheter according to the present invention.

FIG. 2 and FIG. 3 are examples of drug release from coated coupons into an aorta segment according to the formulations of the present invention.

FIG. 4 shows an example of a drug-coated balloon being inflated inside an aorta segment to allow drug transfer into the wall of the aorta.

FIG. 5 shows an example of drug transfer into the aorta wall after the balloon was inflated for 2 minutes to contact the aorta wall and the aorta was opened to expose the inside surface of the aorta.

FIG. 6 shows a graphical representation of a quantitative release of micrograms of a mixture of the pharmaceutical agents rapamycin and curcumin versus time according to an embodiment of the present disclosure.

### DETAILED DESCRIPTION OF EXEMPLARY EMBODIMENTS

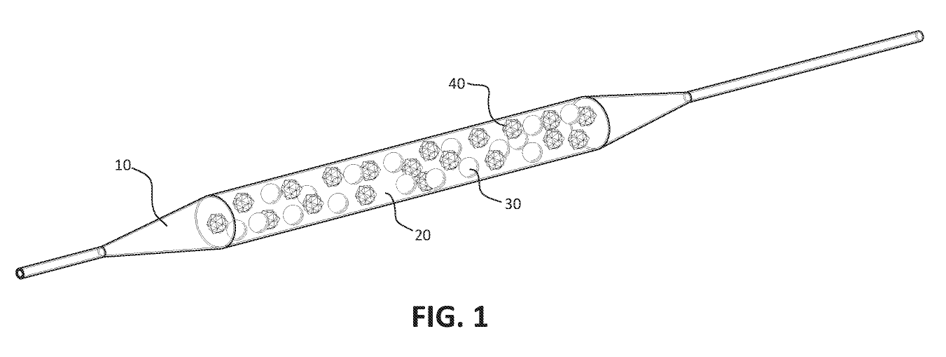

The pharmaceutical compositions described in this invention can be used to coat any surface of medical devices including plastics, metals, ceramic, and biological tissues and parts. As shown in FIG. 1, the medical device is a balloon. The balloon 10 is usually fabricated from plastic materials such as polyethylene, polypropylene, nylon, ethylene vinyl acetate and polyethylene terephthalate (PET). The coating formulation 20 consists of the first drug 30 and the second drug 40 in a proportional ratio. The first drug 30 is an mTor inhibitor and the second drug 40 is an NF-kβ inhibitor. The first drug 30 is rapamycin and the second drug 40 is curcumin. The ratio of rapamycin to curcumin is 3:1.

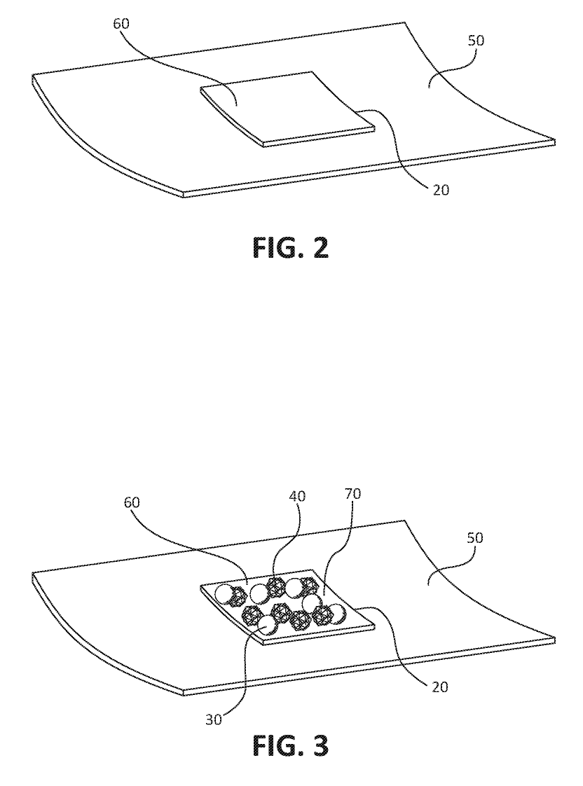

As shown in FIG. 2 and FIG. 3, the coating formulation 20 is coated on a piece of aluminum coupon 60. The coupon is then placed onto the inner surface of an opened aorta 50 with the drug surface in contact with inner surface of the aorta 50. After a few minutes, the coupon 60 is removed showing the area 70 of the aorta where the combination of drug 30 and drug 40 being transferred to the tissue of the aorta 50.

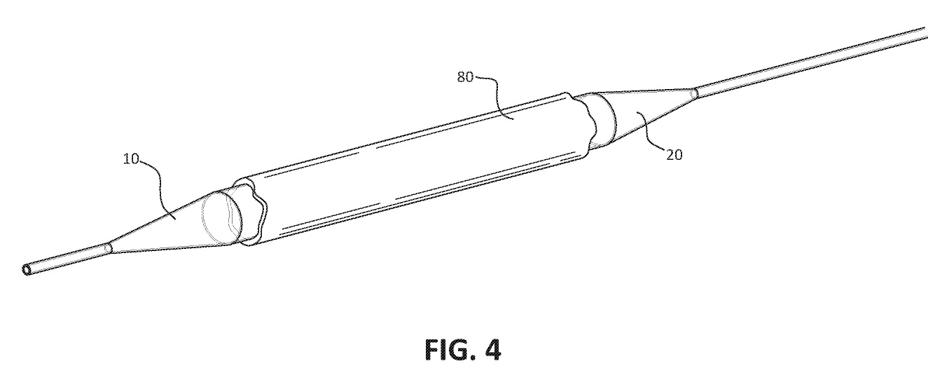

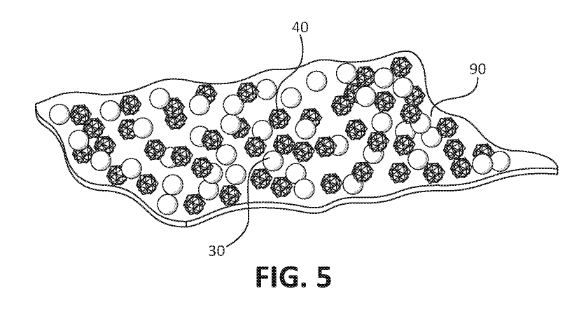

As shown in FIG. 4 and FIG. 5, a balloon 10 with a formulation coating 20 of this invention is being inflated inside an aorta 80 for a maximum time of two minutes. The aorta 80 is then cut opened exposing the surface 90 and showing the transferred of drug 30 and drug 40 into the inner layer of the aorta 80. The drug 30 is an mTor inhibitor and the drug 40 is an NF-kβ inhibitor. In this example, the mTor inhibitor is rapamycin and the NF-kβ inhibitor is curcumin. The ratio of rapamycin to curcumin is 3 to 1. It is advisable that any combination of an mTor inhibitor and an NF-kβ inhibitor can be used and with any ratio depending on the application of each proliferative disease.

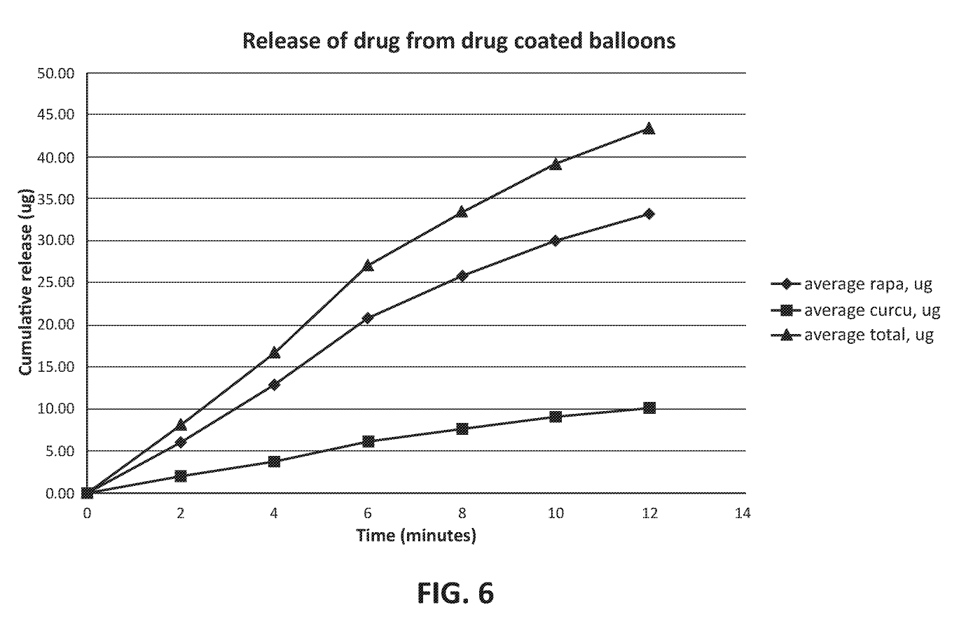

FIG. 6 is a graphical representation of the cumulative releases, in micrograms, of individual NF-kβ inhibitor curcumin and mTor inhibitor rapamycin (first and second curve from the time axis. The third curve from the time axis is the combine total release of both drugs rapamycin and curcumin in micrograms. As shown in FIG. 6 the ratio of rapamycin release is roughly three times the rate of the release of curcumin as verified by the drug ratio of 3 to 1 in the formulation of the coating applied to a device embodiment of the present disclosure.

Embodiments of the present invention relate to medical devices, including particularly balloon catheters and stents, having a rapid drug-releasing coating and methods for preparing such coated devices. The therapeutic agent according to embodiments of the present invention does not require a delayed or long term release and instead is released in a very short time period, from seconds to minutes, to provide a therapeutic effect upon contact with tissue. An object of embodiments of the present invention is to facilitate rapid and efficient uptake of drug by target tissue during transitory device deployment at a target site.

In embodiments of the present invention, dipping and roller coating are the preferred methods because these processes allow one to control the uniformity of the thickness of the coating layer as well as the concentration of the therapeutic agent applied to the medical device. In addition, the operation is safer and less wasteful of materials, especially the pharmaceutical agents. Spraying might also be used but requires the use of more sophisticated equipment such as ultrasonic sprayers and isolators for potent compounds.

In embodiments of this invention, a single coating or multiple coatings can be applied to the intended medical device depending on the concentration of the total drugs in the formulation. The thickness of each coating might vary from about 0.1 microns to 100 microns in thickness depending on the number of dippings or drug concentrations in the formulation.

The following examples include embodiments of medical devices and coating layers within the scope of the present invention. While the following examples are considered to embody the present invention, the examples should not be interpreted as limitations upon the present invention.

Example Preparations of Coating Solutions Include the Following:

Formulation 1: 92.5 mg of rapamycin and 37.3 mg of curcumin in 6.5 ml of the organic solvent tetrahydrofuran (THF). The ratio of rapamycin to curcumin is about 3 to 1 by weight.

Formulation 2: 105.4 mg of rapamycin, and 108.7 mg of curcumin in 7.0 ml tetrahydrofuran (THF). The ratio of rapamycin to curcumin is about 1 to 1 by weight.

Formulation 3: 31.05 mg of rapamycin, 10.35 mg of curcumin and 3.60 mg of citric acid in 6.0 ml tetrahydrofuran (THF). The ratio of rapamycin to curcumin is about 3 to 1 by weight.

Formulation 4: 31.05 mg of rapamycin, 10.35 mg of curcumin and 3.82 mg of Dodecyl methyl sulfoxide (DMSO) in 6.0 ml tetrahydrofuran (THF). The ratio of rapamycin to curcumin is about 3 to 1 by weight.

Formulation 5: 45 mg of polymers (50% polyurethane, 50% polyacrylic acid), 31.05 mg rapamycin, 10.35 mg curcumin in 6.0 ml THF. The ratio of rapamycin to curcumin is 3 to 1.

What is claimed is:

1. A method for treating proliferative diseases by delivering a combination of at least two pharmaceutically active agents to a diseased area or tissue comprising:
a coating layer of two hydrophobic drugs;
applied to an exterior surface of a device or a substrate;
wherein the first pharmaceutically active agent is selected from a group consisting of mTor inhibitors and the second pharmaceutically active agent is selected from a group of consisting of NF-kβ inhibitors.

2. The method of claim 1, wherein the mTor inhibitor is rapamycin and the NF-kβ inhibitor is curcumin.

3. The method of claim 1, wherein the mTor inhibitor is rapamycin and the NF-kβ inhibitor is selected from a group consisting of: sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, and cycloepoxydon tepoxalin and combinations thereof.

4. The method of claim 1, wherein the ratio by weight of the mTor inhibitor to the NF-kβ inhibitor in the coating layer is from 1:1 to 100:1.

5. The method of claim 1, wherein the combined initial drug loading is from 0.1 micrograms to 10 micrograms of pharmaceutically active agents per square millimeter of the device or substrate.

6. The method of claim 3, wherein the NF-kβ inhibitor is selected from a group consisting of: sulfasalazine, indomethacin, minocycline, rifampin and a combination thereof.

7. A method for treating proliferative diseases by delivering a combination of at least two pharmaceutically active therapeutic agents to a diseased area or tissue comprising:
a coating layer applied to an exterior surface of two hydrophobic drugs on the exterior surface of a device or substrate, containing;
a permeation enhancer;
a combination of at least two pharmaceutically active therapeutic agents;
wherein the first pharmaceutically active therapeutic agent is selected from a group consisting of mTor inhibitors and the second pharmaceutically active therapeutic agent is selected from a group of consisting of NF-kβ inhibitors.

8. The method of claim 7, wherein the permeation enhancer is citric acid.

9. The method of claim 7, wherein the permeation enhancer is dodecyl methyl sulfoxide (DMSO).

10. The method of claim 7, wherein the permeation enhancer is L-arginine.

11. The method of claim 7, wherein the permeation enhancer is sodium nitroprusside.

12. The method of claim 7, wherein the permeation enhancer is a nitric oxide (NO) donor.

13. The method of claim 7, wherein the permeation enhancer is selected from a group of S-nitrosothiols consisting of S-nitroso-N-acetylamine (SNAP), S-nitrosoglutathione (SNOGLU) and S-nitroso-N-valerylpenicillamine (SNVP) and a group of Diazeniumdiolates (NONOates) consisting of Diethyamino NONOate (DEA-NO), PROLI/NO, SPER/NO and V-PYRRO/NO.

14. A method for treating proliferative diseases by delivering a combination of at least two pharmaceutically active agents to a diseased area or tissue comprising:
a coating layer of two hydrophobic drugs applied to an exterior surface of a medical device or substrate;
a polymer blend carrier for the pharmaceutically active agents;
wherein a first pharmaceutically active agent is selected from a group consisting of mTor inhibitors and a second pharmaceutically active agent is selected from a group consisting of NF-kβ inhibitors.

15. The method of claim 14, wherein the polymer blend carrier is a mixture of hydrophilic polyurethane and a polyacrylic polymer.

16. The method of claim 14, wherein the weight ratio of the polyurethane polymer to the polyacrylic polymer in the polymer blend carrier is from 1:1 to 10:1.

17. The method of claim 14, wherein the weight percentage of pharmaceutically active agents to the total weight of the polymer blend carrier is from 30% to 70%.

18. The method of claim 14, wherein the mTor inhibitor is rapamycin and the NF-kβ inhibitor is curcumin.

19. The method of claim 14, wherein the mTor inhibitor is rapamycin and the NF-kβ inhibitor is selected from a group consisting of: sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, and cycloepoxydon tepoxalin and mixtures thereof.

20. The method of claim 14, wherein the ratio by weight of the mTor inhibitor in the coating layer to the NF-kβ inhibitor is from 1:1 to 100:1.

21. The method of claim 14, wherein the combined initial drug loading is from 0.1 micrograms to 10 micrograms of therapeutic agents per square millimeter of the device or substrate.
